(19) United States
(12) Patent Application Publication
(10) Pub. No.: US 2009/0250715 A1
(43) Pub. Date: Oct. 8, 2009

(54) LED TRANSPARENT BRICK

(75) Inventors: TSE AN LEE, TAINAN CITY (TW); CHIH MING LAI, PINGTUNG CITY (TW)

Correspondence Address: WPAT, PC;INTELLECTUAL PROPERTY ATTORNEYS, 2030 MAIN STREET, SUITE 1300, IRVINE, CA, 92614, US

(73) Assignee: FOXSEMICON INTEGRATED TECHNOLOGY INC., MIAOLI COUNTY (TW)

(21) Appl. No.: 12/331,935

(22) Filed: Dec. 10, 2008

(30) Foreign Application Priority Data
Apr. 2, 2008 (TW) 097111941

(51) Int. Cl. H01L 33/00

(52) U.S. Cl. 257/98; 257/E33.067; 257/E33.068

(57) ABSTRACT

An LED transparent brick comprises a transparent brick body, a photo catalyst layer, a connecting layer, a light reflection layer and one or more ultraviolet LEDs. The photo catalyst layer is placed at a first surface of the transparent brick body, and the connecting layer combines the transparent brick body and the photo catalyst layer. The light reflection layer is placed at a second surface of the transparent brick body, and the second surface corresponds to the first surface. The ultraviolet LED is placed at a side of the transparent brick body, and illuminates the photo catalyst layer to generate oxidative decomposition and hydrophilic capabilities. The light reflection layer can increase light reflection in the transparent brick body to the photo catalyst layer.

### BACKGROUND OF THE INVENTION

(A) Field of the Invention

The present invention is related to a transparent brick, and more specifically, to a light-emitting diode (LED) transparent brick.

(B) Description of the Related Art

Normally, bricks are made of ceramic clay, feldspar or quartz, and are fabricated by a high-temperature process. Bricks are placed onto inner or outer walls, ground and the surfaces of buildings for the purpose of decoration. The bricks placed in easily contaminated environments, such as bathrooms or kitchens, may have germs or greasy dirt on them. It is necessary to maintain a sterile environment in hospitals that make diagnoses, prescribe medication and perform surgical operations, so the bricks must be cleaned from time to time to remove germs and dirt.

Photo catalyst surfacing features high oxidation decomposition capability and is hydrophilic after being illuminated by ultraviolet light. The high oxidation decomposition capability can decompose dirt, germs and organic grease adhered to the photo catalyst surface. The so-called “hydrophilic” is related to the surface of the photo catalyst, on which water does not accumulate into drops, and instead are dispersed. Accordingly, water can permeate between the photo catalyst surface and dirt and grease to easily remove the dirt and grease.

Taiwan Patent No. M308955 proposed an antiseptic Mosaic glass brick. The glass brick is daubed with a cured antiseptic layer. The cured antiseptic layer includes curing polymer and titanium dioxide grains. The titanium dioxide grains are distributed into the antiseptic layer. The photo catalyst is surface-active; dirt is decomposed only when it contacts the photo catalyst directly. If the photo catalyst is in the form of a grain and is distributed in the antiseptic layer, the photo catalyst cannot make direct contact with the dirt. Consequently, the decomposition efficacy to dirt is limited. In addition, the antiseptic Mosaic glass brick does not provide ultraviolet light, and ultraviolet light is but a small portion of sunlight, so the photo catalyst cannot work efficiently. If the glass brick is placed indoors, there is no ultraviolet light for the photo catalyst reaction and thus the antiseptic function becomes ineffective. Therefore, there is a demand to provide bricks with a self-cleaning feature for the applications in which cleanliness needs to be maintained for a long time.

### SUMMARY OF THE INVENTION

The present invention is directed to providing a transparent brick with self-cleaning and sterilization functions by using the high oxidation decomposition and hydrophilic capabilities offered by photo-catalyst surface when it is illuminated by ultraviolet light, so as to provide a self-cleaning function on the transparent brick surface. The high oxidation decomposition capability can decompose grease, germs and viruses, causing the subjects adhered to the transparent brick to easily come off. Therefore, the self-cleaning brick with photo catalyst surface can remove dirt and have antiseptic efficacy.

In accordance with the present invention, an LED transparent brick comprises a transparent brick body, a photo catalyst layer, a connecting layer, a light reflection layer and one or more ultraviolet LEDs. The photo catalyst layer is placed at a first surface of the transparent brick body, and the connecting layer combines the transparent brick body and the photo catalyst layer. The light reflection layer is placed at a second surface of the transparent brick body, and the second surface corresponds to the first surface. The ultraviolet LED is placed at a side of the transparent brick body, and illuminates the photo catalyst layer to generate oxidative decomposition and hydrophilic capabilities. The light reflection layer can increase light reflection in the transparent brick body to the photo catalyst layer.

In an embodiment, titanium dioxide photo catalyst is formed on a surface of the transparent brick body. The ultraviolet light generated by UV-LED penetrates through the transparent brick body and the connecting layer to the photo catalyst layer, and illuminates the titanium dioxide photo catalyst to generate high oxidative decomposition capability to decompose the subjects such as grease or germs on the photo catalyst layer. Moreover, the transparent brick surface illuminated by ultraviolet light is hydrophilic and therefore has self-cleaning capability.

The LED transparent brick of the present invention, which can maintain surface cleanliness for a long time, may be applied to walls or ceilings, and can be shaped for upholstery.

The photo catalyst on the surface of the LED transparent brick is in direct contact with surface dirt, and is illuminated by ultraviolet light to achieve self-cleaning function and sterilization.

### BRIEF DESCRIPTION OF THE DRAWINGS

FIG. 1 illustrates an LED transparent brick in accordance with an embodiment of the present invention;

FIGS. 2A and 2B illustrate transparent brick bodies of the LED transparent bricks in accordance with the present invention;

FIGS. 3A through 3C illustrate LED transparent bricks in accordance with the present invention;

FIGS. 4A and 4B illustrate the LED arrangement of the LED transparent bricks in accordance with the present invention;

FIGS. 5 and 6 illustrate the oxidation decomposition and hydrophilic dirt removal mechanisms of the LED transparent bricks in accordance with the present invention; and

FIG. 7 illustrates an LED transparent brick in accordance with another embodiment in accordance with the present invention.

### DETAILED DESCRIPTION OF THE INVENTION

The present invention is explained with reference to drawings below, so as to clearly describe the features of the present invention.

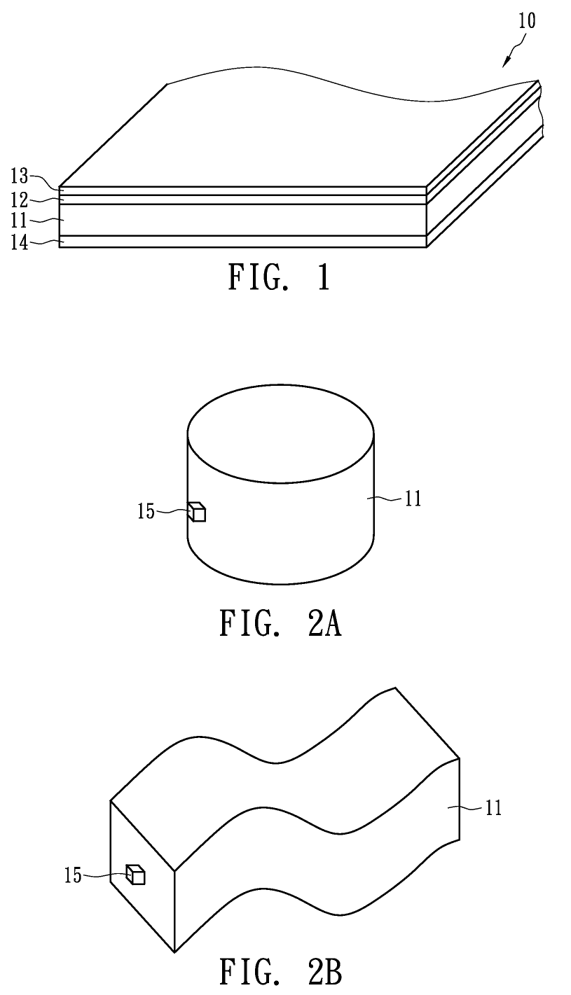

FIG. 1 illustrates a transparent brick 10 in accordance with a first embodiment of the present invention. The transparent brick 10 may comprise a transparent brick body 11, a connecting layer 12, a photo catalyst layer 13 and a light reflection layer 14. The transparent brick body 11 may be made of hard transparent plastic material such as polymethylmethacrylate (PMMA) or polycarbonate (PC), or quartz, or tempered glass. The light reflection layer 14 can reflect light from the interior of the transparent brick body 11 to the photo catalyst layer 13. The light reflection layer 14 is placed at a non-emitting surface. The transparent brick body 11 may be in the form of a cube, cylinder or curved cube as shown in FIG. 2A and FIG. 2B.

The connecting layer 12 is formed on a surface of the transparent brick body 11 to combine the transparent brick body 11 and the photo catalyst layer 13 and to prevent the transparent brick body 11 from being eroded by oxidative decomposition when the photo catalyst layer 13 is illuminated by ultraviolet light. The connecting layer 12 may be made of silicon dioxide or polytetrafluoroethene (PTFE), i.e., Teflon™.

The photo catalyst layer 13 is formed on the connecting layer 12, so that the photo catalyst layer 13 can directly contact and decompose dirt. In an embodiment, the photo catalyst layer 13 may include titanium dioxide (TiO₂). In practice, other photo catalyst materials including oxide such as zinc oxide (ZnO), tin dioxide (SnO₂) and zirconium dioxide (ZrO₂), or sulfide such as cadmium sulfide (CdS) and zinc sulfide (ZnS) may be used instead.

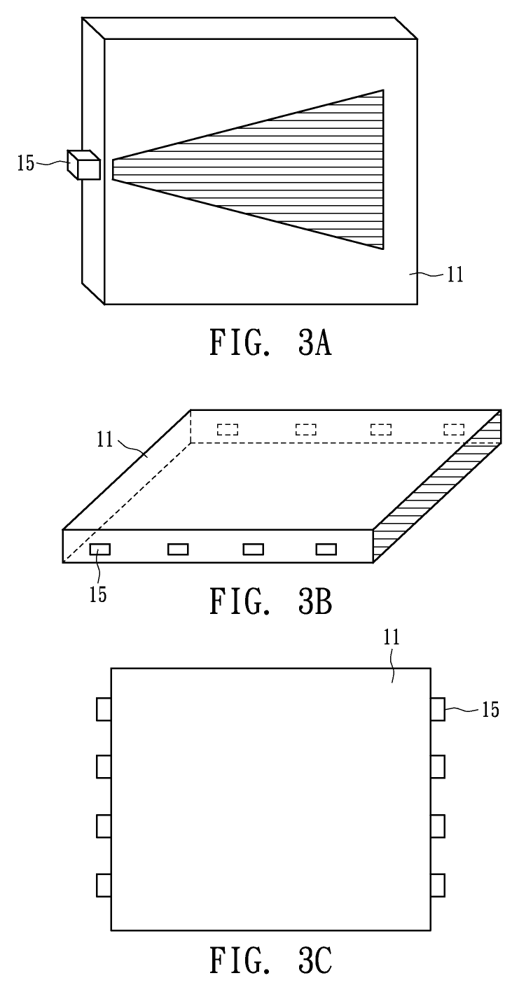

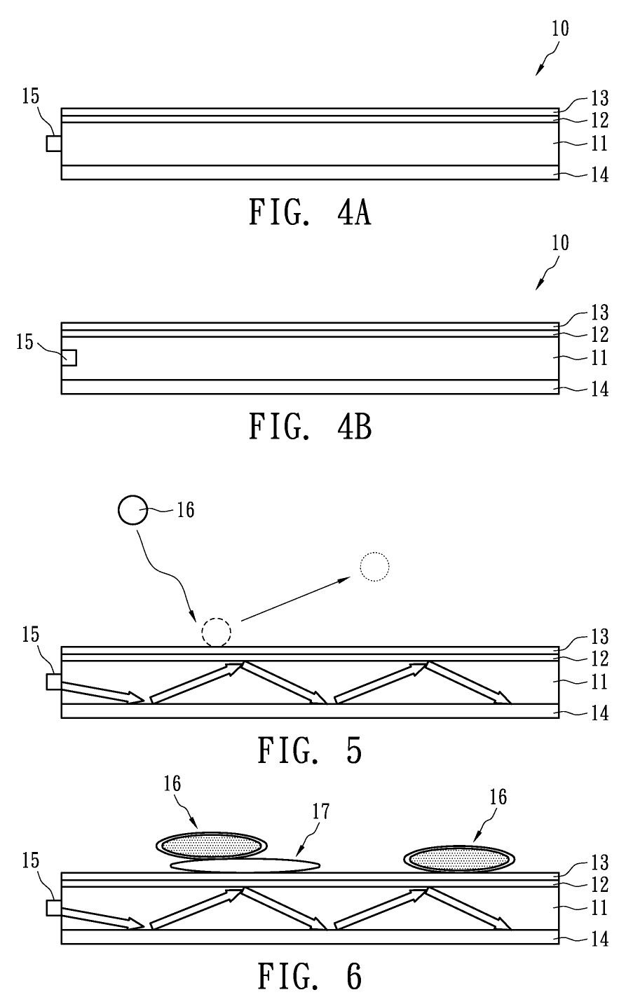

According to an embodiment of the present invention, an ultraviolet LED (UV-LED) 15 is used as an ultraviolet light source. The UV-LED 15 is placed at a side of the transparent brick body 11 in a side-view manner. The UV-LED 15 may be a point light source (single LED device) as shown in FIG. 3A, or a light row (plural LED devices) placed at a side as shown in FIGS. 3B and 3C. The UV-LED 15 can be placed at the outside of the transparent brick body 11 as shown in FIG. 4A. Alternatively, a portion of the transparent brick body 11 may be removed so that the UV-LED 15 can be placed at the inside of the transparent brick body 11 as shown in FIG. 4B.

Referring to FIG. 5, the UV-LED 15 emits ultraviolet light that penetrates through the transparent brick body 11 and reaches the photo catalyst layer 13. The photo catalyst layer 13, e.g., a TiO₂ layer, absorbs ultraviolet light to generate high oxidative decomposition capability on the surface thereof, whereby small molecules such as germs or other tiny dirt particles 16 on the surface of the photo catalyst layer 13 will be decomposed.

As shown in FIG. 6, TiO₂ is exemplified as the photo catalyst layer 13, which absorbs ultraviolet light and becomes hydrophilic. If dirt 16 adheres to the surface of TiO₂, water 17 can permeate between the dirt 16 and the photo catalyst layer 13, thereby removing the dirt 16.

In an embodiment, the UV-LED 15 may have a wavelength below 380 nm, i.e., near ultraviolet light, for being absorbed by the photo catalyst. To avoid damage to DNA of human bodies caused by ultraviolet light having short wavelength, UV-LED 15 may use an ultraviolet LED of a wavelength between 350 and 380 nm.

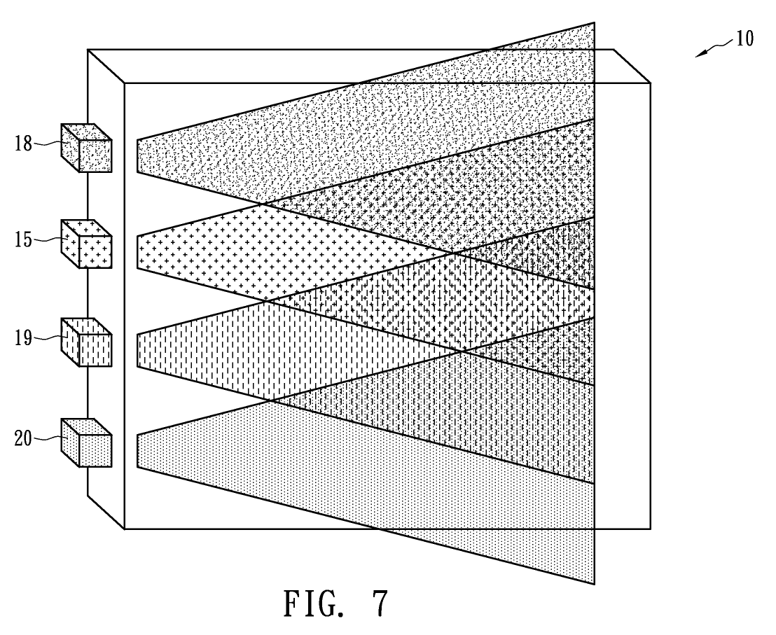

As shown in FIG. 7, to decorate the transparent brick, a side of the transparent brick 10 may be patterned or shaped, or equipped with a red LED 18, a yellow LED 19 or a blue LED 20 to enrich the colors of the transparent brick 10 and thus enhance visual effect.

In this embodiment, the photo catalyst transparent brick associated with UV-LED has surface-sterilization, dirt removal and surface-cleaning functions, and can be used in dirty places such as bathrooms or kitchens, or high sterility environments such as hospitals. Moreover, the transparent brick can be associated with LEDs of various colors to create colorful bricks, and can be applied to upholstery on walls or ceilings of buildings.

The above-described embodiments of the present invention are intended to be illustrative only. Numerous alternative embodiments may be devised by those skilled in the art without departing from the scope of the following claims.

What is claimed is:

1. An LED transparent brick, comprising:
a transparent brick body
a photo catalyst layer placed at a first surface of the transparent brick body;
a connecting layer combining the transparent brick body and the photo catalyst layer;
a light reflection layer placed at a second surface of the transparent brick body, the second surface corresponding to the first surface; and
at least one ultraviolet LED placed at a side of the transparent brick body, the ultraviolet LED illuminating the photo catalyst layer to generate oxidative decomposition and hydrophilic capabilities.

2. The LED transparent brick in accordance with claim 1, wherein the ultraviolet LED emits light having a wavelength less than 380 nm.

3. The LED transparent brick in accordance with claim 1, wherein the ultraviolet LED emits light having a wavelength between 350 and 380 nm.

4. The LED transparent brick in accordance with claim 1, wherein a plurality of ultraviolet LEDs are placed in array manner.

5. The LED transparent brick in accordance with claim 1, wherein the transparent brick body is made of polymethylmethacrylate, polycarbonate, quartz or glass.

6. The LED transparent brick in accordance with claim 1, wherein the transparent brick body is in the shape of cube, cylinder or curved cube.

7. The LED transparent brick in accordance with claim 1, wherein the transparent brick body is patterned or shaped.

8. The LED transparent brick in accordance with claim 1, further comprising LEDs of different colors on the transparent brick body.

9. The LED transparent brick in accordance with claim 8, wherein the LEDs are selected from red, blue, yellow or green LEDs.

10. The LED transparent brick in accordance with claim 1, wherein the connecting layer is made of silicon dioxide or polytetrafluoroethene.

11. The LED transparent brick in accordance with claim 1, wherein the light reflection layer is placed at a non-emitting surface of the transparent brick body.

12. The LED transparent brick in accordance with claim 1, wherein the photo catalyst layer is made from the group consisting of titanium dioxide, zinc oxide, tin dioxide, zirconium dioxide, cadmium sulfide and zinc sulfide.

13. The LED transparent brick in accordance with claim 1 being placed on walls or ceilings.
